(19) United States
(12) Patent Application Publication
(10) Pub. No.: US 2006/0148744 A1
(43) Pub. Date: Jul. 6, 2006

(54) Use of cis-epoxyeicosantrienoic acids and inhibitors of soluble epoxide hydrolase to reduce damage from stroke

(75) Inventors: Bruce D. Hammock, Davis, CA (US); John D. Imig, Evans, GA (US); Anne M. Dorrance, Thomson, GA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: REGENTS OF THE UNIVERSITY OF CALIFORNIA, Oakland, CA (US); MEDICAL COLLEGE OF GEORGIA, Augusta, GA (US)

(21) Appl. No.: 11/234,845

(22) Filed: Sep. 23, 2005

Related U.S. Application Data
(60) Provisional application No. 60/612,906, filed on Sep. 23, 2004.

(51) Int. Cl. A61K 48/00; A61K 31/336; A61K 31/17

(52) U.S. Cl. 514/44; 514/475; 514/588

(57) ABSTRACT

The invention provides uses and methods for reducing brain damage from stroke. The uses comprise the use of an inhibitor of soluble epoxide hydrolase (sEH) for the manufacture of a medicament to reduce brain damage from stroke, as well as the use of cis-epoxyeicosatrienoic acid (EET) for that purpose. The methods comprise the administration of sEH inhibitors to persons who have had a stroke, or who are at risk of having a stroke. Optionally, the methods also include the administration of EETs.

### STATEMENT AS TO RIGHTS TO INVENTIONS MADE UNDER FEDERALLY SPONSORED RESEARCH AND DEVELOPMENT

This invention was made with government support under Grant Nos. DK38226, ES02710, ES05707 and HL59699, awarded by the National Institutes of Health. The Government has certain rights in this invention.

### CROSS-REFERENCES TO RELATED APPLICATIONS

This application claims priority to U.S. Ser. No. 60/612,906, filed Sep. 23, 2004, which is incorporated in its entirety by this reference.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

According to the American Heart Association, stroke is the third leading cause of death in the United States, and a leading cause of long term disability. Stroke is a disorder of the blood vessels that supply the brain. There are two main types of stroke: ischemic, in which a blood vessel is blocked, and hemorrhagic, in which a vessel bleeds into the brain, exerting pressure on the surrounding brain tissue. According to the American Stroke Association (“ASA”) website, some 88% of strokes are ischemic; thus, ischemic strokes are by far the most common stroke event.

The ASA further indicates that the risk factors for stroke include the following: high blood pressure, defined as blood pressure of 140/90 or higher, tobacco use, diabetes, carotid artery disease, peripheral artery disease, atrial fibrillation, transient ischemic attacks (TIAs), blood disorders such as high red blood cell counts and sickle cell disease, high blood cholesterol, obesity, alcohol use of more than one drink a day for women or two drinks a day for men, use of cocaine, a family history of stroke, a previous stroke or heart attack, and being elderly. Stroke is also more common in men, but more women then men die from stroke.

Epoxide hydrolases (EHs) are enzymes that add water to epoxides resulting in their corresponding 1,2-diols (Hammock, B. D. et al., in Comprehensive Toxicology: Biotransformation (Elsevier, N.Y.), pp. 283-305 (1997); Oesch, F. Xenobiotica 3:305-340 (1972)). Four principal EH's are known: leukotriene epoxide hydrolase, cholesterol epoxide hydrolase, microsomal EH (“mEH”), and soluble EH (“sEH,” previously called cytosolic EH). The leukotriene EH acts on leukotriene A₄, whereas the cholesterol EH hydrates compounds related to the 5,6-epoxide of cholesterol (Nashed, N. T., et al., Arch. Biochem. Biophysics., 241:149-162, 1985; Finley, B. and B. D. Hammock, Biochem. Pharmacol., 37:3169-3175, 1988). The microsomal epoxide hydrolase metabolizes monosubstituted, 1,1-disubstituted, cis-1,2-disubstituted epoxides and epoxides on cyclic systems epoxides to their corresponding diols. Because of its broad substrate specificity, this enzyme is thought to play a significant role in ameliorating epoxide toxicity. Reactions of detoxification typically decrease the hydrophobicity of a compound, resulting in a more polar and thereby excretable substance.

Soluble EH is only very distantly related to mEH and hydrates a wide range of epoxides not on cyclic systems. In contrast to the role played in the degradation of potential toxic epoxides by mSH, sEH is believed to play a role in the formation or degradation of endogenous chemical mediators. For instance, cytochrome P450 epoxygenase catalyzes NADPH-dependent enatioselective epoxidation of arachidonic acid to four optically active cis-epoxyeicosantrienoic acids (“EETs”) (Karara, A., et al., J. Biol. Chem., 264:19822-19877, (1989)). Soluble epoxide hydrolase has been shown in vivo to convert these compounds with regio- and enantiofacial specificity to the corresponding vic-dihydroxyeicosatrienoic acids (“DHETs”). Both liver and lung cytosolic fraction hydrolyze 14,15-EET, 8,9-EET and 11,12-EET, in that order of preference. The 5,6 EET is hydrolyzed more slowly. Purified sEH selects 8S,9R- and 14R,15S-EET over their enantiomers as substrates. Studies have revealed that EETs and their corresponding DHETs exhibit a wide range of biological activities. Some of these activities include involvements in luteinizing hormone-releasing hormone, stimulation of luteinizing hormone release, inhibition of Na⁺/K⁺ ATPase, vasodilation of coronary artery, mobilization of Ca²⁺ and inhibition of platelet aggregation.

It would be desirable to be able to reduce the damage from stroke.

### BRIEF SUMMARY OF THE INVENTION

In a first group of embodiments, the invention provides uses of one or more inhibitors of soluble epoxide hydrolase (“sEH”) for the manufacture of a medicament to reduce brain damage from stroke. The stroke can be ischemic stroke or hemorrhagic stroke. In some embodiments, the inhibitor of sEH is adamantyl dodecyl urea, N-cyclohexyl-N′-dodecyl urea (CDU) or N,N′-dicyclohexylurea (DCU). In a preferred embodiment, the inhibitor is adamantyl dodecanoic acid (AUDA.

The invention further provides the use of one or more cis-epoxyeicosantrienoic acids (“EETs”) for the manufacture of a medicament to reduce brain damage from stroke. The EET can be, for example, 14,15-EET, 8,9-EET, or 11,12-EET.

In additional embodiments, the invention provides the use of a nucleic acid that inhibits expression of soluble epoxide hydrolase (“sEH”) for the manufacture of a medicament for reducing brain damage from stroke. In some embodimens, the nucleic acid is a small interfering RNA.

In yet additional embodiments, the invention provides methods of reducing brain damage from a stroke, comprising administering an inhibitor of soluble epoxide hydrolase (“sEH”) to a subject who has suffered a stroke. In some preferred embodiments, the sEH inhibitor is administered within 6 hours of said stroke. In some embodiments, it is administered within 3 hours or less of a stroke. The sEH inhibitor can be, for example, adamantyl dodecyl urea (particularly adamantyl dodecanoic acid), N-cyclohexyl-N′-dodecyl urea (CDU) and N,N′-dicyclohexylurea (DCU). The method can further comprise administering a cis-epoxyeicosantrienoic acid (“EET”) to the subject. The EET can be, for example, 14,15-EET, 8,9-EET or 11,12-EET.

In another group of embodiments, the invention provides methods of reducing brain damage from a stroke, comprising administering an inhibitor of soluble epoxide hydrolase (“sEH”) to a subject at risk of suffering a stroke. The subject can be a person who has hypertension, a person who uses tobacco, a person who has carotid artery disease, a person who has peripheral artery disease, a person who has atrial fibrillation, a person who has had one or more transient ischemic attacks (TIAs), a person who has a high red blood cell count, a person who has sickle cell disease, a person who has high blood cholesterol, a person who is obese, a female who uses alcohol in excess of one drink a day, a male who uses alcohol in excess of two drinks a day, a person who uses cocaine, a person who has a family history of stroke, a person who has had a previous stroke or heart attack, a person who has diabetes, or a person who is 60 years or more of age. Some individuals can have two or more of these factors. The sEH inhibitor can be, for example, adamantyl dodecanoic acid, N-cyclohexyl-N′-dodecyl urea (CDU) and N,N′-dicyclohexylurea (DCU). The methods can further include administering a cis-epoxyeicosantrienoic acid (“EET”) to the subject. The EET can be, for example, 14,15-EET, 8,9-EET or 11,12-EET. The sEH inhibitor can also be a nucleic acid which inhibits expression of a gene encoding sEH. The nucleic acid can be a short interfering RNA (“siRNA”).

### DETAILED DESCRIPTION OF THE INVENTION

I. Introduction

A. Prophylatic and Therapeutic Use of sEH Inhibitors and EETS to Reduce Stroke Damage

Surprisingly, it has now been discovered that inhibitors of soluble epoxide hydrolase (“sEH”) and EETs administered in conjunction with inhibitors of sEH can reduce brain damage from strokes.

In the studies reported in the Examples, an exemplar sEH inhibitor was administered to stroke prone, spontaneously hypertensive rats (“SPSHR”) for six weeks and an ischemic stroke was then mimicked. Animals to which the exemplar sEH inhibitor had been administered had a significantly decreased area of brain damage compared to animals to which the sEH inhibitor had not been administered. Based on these results, we expect that inhibitors of sEH taken prior to an ischemic stroke will reduce the area of brain damage and will likely reduce the consequent degree of impairment. The reduced area of damage should also be associated with a faster recovery from the effects of the stroke.

While the pathophysiologies of different subtypes of stroke differ, they all cause brain damage. Hemorrhagic stroke differs from ischemic stroke in that the damage is largely due to compression of tissue as blood builds up in the confined space within the skull after a blood vessel ruptures, whereas in ischemic stroke, the damage is largely due to loss of oxygen supply to tissues downstream of the blockage of a blood vessel by a clot. Ischemic strokes are divided into thrombotic strokes, in which a clot blocks a blood vessel in the brain, and embolic strokes, in which a clot formed elsewhere in the body is carried through the blood stream and blocks a vessel there. But, in both hemorrhagic stroke and ischemic stroke, the damage is due to the death of brain cells. Based on the results observed in our studies, however, we would expect at least some reduction in brain damage in all types of stroke and in all subtypes.

As noted in the Background, there are a number of factors associated with an increased risk of stroke. Given the results of the studies underlying the present invention, sEH inhibitors administered to persons with any one or more of the following conditions or risk factors:high blood pressure, tobacco use, diabetes, carotid artery disease, peripheral artery disease, atrial fibrillation, transient ischemic attacks (TIAs), blood disorders such as high red blood cell counts and sickle cell disease, high blood cholesterol, obesity, alcohol use of more than one drink a day for women or two drinks a day for men, use of cocaine, a family history of stroke, a previous stroke or heart attack, or being elderly, will reduce the area of brain damaged of a stroke. With respect to being elderly, the risk of stroke increases for every 10 years. Thus, as an individual reaches 60, 70, or 80, administration of sEH inhibitors has an increasingly larger potential benefit. As noted in the next section, the administration of EETs in combination with one or more sEH inhibitors can be beneficial in further reducing the brain damage.

In some preferred uses and methods, the sEH inhibitors and, optionally, EETs, are administered to persons who use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

Clot dissolving agents, such as tissue plasminogen activator (tPA), have been shown to reduce the extent of damage from ischemic strokes if administered in the hours shortly after a stroke. tPA, for example, is approved by the FDA for use in the first three hours after a stroke. Thus, at least some of the brain damage from a stoke is not instantaneous, but occurs over a period of time or after a period of time has elapsed after the stroke. It is therefore believed that administration of sEH inhibitors, optionally with EETs, can also reduce brain damage if administered within 6 hours after a stroke has occurred, more preferably within 5, 4, 3, or 2 hours after a stroke has occurred, with each successive shorter interval being more preferable. Even more preferably, the inhibitor or inhibitors are administered 2 hours or less or even 1 hour or less after the stroke, to maximize the reduction in brain damage. Persons of skill are well aware of how to make a diagnosis of whether or not a patient has had a stroke. Such determinations are typically made in hospital emergency rooms, following standard differential diagnosis protocols and imaging procedures.

In some preferred uses and methods, the sEH inhibitors and, optionally, EETs, are administered to persons who have had a stroke within the last 6 hours who: use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

B. Use of EETS in Combination with sEH Inhibitors

EETs are epoxides of arachidonic acid and are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into DHETs is reduced.

EETs have not been administered therapeutically, largely because it has been believed they would be hydrolyzed too quickly by endogenous sEH to be helpful. Based on other work conducted in the labs of the present inventors, it is now believed that endogenous sEH can be inhibited sufficiently to permit administration of exogenous EET to result in increased levels of EETs over those normally present. Thus, the methods of the invention include administration of EETs in combination with one or more sEH inhibitors, or during such time as the sEH inhibitor or inhibitors are at levels sufficient to reduce EET degradation by endogenous sEH.

EETs useful in the methods of the present invention include 14,15-EET, 8,9-EET and 11,12-EET, and 5,6 EETs, in that order of preference. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

Medicaments of EETs can be made which can be administered in conjunction with one or more sEH inhibitors, or a medicament containing one or more sEH inhibitors can optionally contain one or more EETs. The EETs can be administered concurrently with the sEH inhibitor, or following administration of the sEH inhibitor. It is understood that, like all drugs, inhibitors have half lives defined by the rate at which they are metabolized by or excreted from the body, and that the inhibitor will have a period following administration during which it will be present in amounts sufficient to be effective. If EETs are administered after the inhibitor is administered, therefore, it is desirable that the EETs be administered during the period during which the inhibitor will be present in amounts to be effective to delay hydrolysis of the EETs. Typically, the EET or EETs will be administered within 48 hours of administering an sEH inhibitor. Preferably, the EET or EETs are administered within 24 hours of the inhibitor, and even more preferably within 12 hours. In increasing order of desirability, the EET or EETs are administered within 10, 8, 6, 4, 2, hours, 1 hour, or one half hour after administration of the inhibitor. Most preferably, the EET or EETs are administered concurrently with the inhibitor.

In some embodiments, the sEH inhibitor may be a nucleic acid, such as a small interfering RNA (siRNA), which reduces expression of a gene encoding sEH. The EETs may be administered in combination with such a nucleic acid. Typically, a study will determine the time following administration of the nucleic acid before a decrease is seen in levels of sEH. The EET or EETs will typically then be administered a time calculated to be after the activity of the nucleic acid has resulted in a decrease in sEH levels.

In some embodiments, the EETs, the sEH inhibitor, or both, are provided in a material that permits them to be released over time to provide a longer duration of action. Slow release coatings are well known in the pharmaceutical art; the choice of the particular slow release coating is not critical to the practice of the present invention.

EETs are subject to degradation under acidic conditions. Thus, if the EETs are to be administered orally, it is desirable that they are protected from degradation in the stomach. Conveniently, EETs for oral administration may be coated to permit them to passage the acidic environment of the stomach into the basic environment of the intestines. Such coatings are well known in the art. For example, aspirin coated with so-called “enteric coatings” is widely available commercially. Such enteric coatings may be used to protect EETs during passage through the stomach. A exemplar coating is set forth in the Examples.

II. Definitions

Units, prefixes, and symbols are denoted in their Système International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety. Terms not defined herein have their ordinary meaning as understood by a person of skill in the art.

“Epoxide hydrolases” (“EH;” EC 3.3.2.3) are enzymes in the alpha beta hydrolase fold family that add water to 3 membered cyclic ethers termed epoxides.

“Soluble epoxide hydrolase” (“sEH”) is an enzyme which in endothelial and smooth muscle cells converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO:1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)). Unless otherwise specified, as used herein, the terms “soluble epoxide hydrolase” and “sEH” refer to human sEH.

Unless otherwise specified, as used herein, the term “sEH inhibitor” refers to an inhibitor of human sEH. Preferably, the inhibitor does not also inhibit the activity of microsomal epoxide hydrolase by more than 25% at concentrations at which the inhibitor inhibits sEH by at least 50%, and more preferably does not inhibit mEH by more than 10% at that concentration. For convenience of reference, unless otherwise required by context, the term “sEH inhibitor” as used herein encompasses prodrugs which are metabolized to active inhibitors of sEH. Further for convenience of reference, and except as otherwise required by context, reference herein to a compound as an inhibitor of sEH includes reference to derivatives of that compound (such as an ester of that compound) that retain activity as an sEH inhibitor.

“cis-Epoxyeicosatrienoic acids” (“EETs”) are biomediators synthesized by cytochrome P450 epoxygenases.

“Stroke” has been defined as a clinical syndrome characterized by rapidly developing clinical signs of focal (or global) disturbance lasting 24 hours or longer or leading to death with no apparent cause other than of vascular origin. The definition of stroke incorporates hemorrhagic and ischemic lesions, with additional subtypes in both categories.

“Hemorrhagic stroke” is a stroke resulting from the rupture or leakage of a blood vessel in the brain.

“Ischemic stroke” is a stroke resulting from a blockage of a blood vessel in the brain by a clot.

By “physiological conditions” is meant an extracellular milieu having conditions (e.g., temperature, pH, and osmolarity) which allows for the sustenance or growth of a cell of interest.

Unless otherwise required by context, “administering” an EET and an sEH inhibitor to a person in need thereof includes administering an sEH inhibitor, followed by a later administration of an EET while an amount of sEH inhibitor is still present sufficient to reduce by at least 25% the rate of hydrolysis of the EET by sEH.

III. Inhibitors of Soluble Epoxide Hydrolase

Scores of sEH inhibitors are known, of a variety of chemical structures. Derivatives in which the urea, carbamate, or amide pharmacophore (as used herein, “pharmacophore” refers to the section of the structure of a ligand that binds to the sEH) is covalently bound to both an adamantane and to a 12 carbon chain dodecane are particularly useful as sEH inhibitors. Derivatives that are metabolically stable are preferred, as they are expected to have greater activity in vivo. Selective and competitive inhibition of sEH in vitro by a variety of urea, carbamate, and amide derivatives is taught, for example, by Morisseau et al., Proc. Natl. Acad. Sci. U.S. A, 96:8849-8854 (1999), which provides substantial guidance on designing urea derivatives that inhibit the enzyme.

Derivatives of urea are transition state mimetics that form a preferred group of sEH inhibitors. Within this group, N,N′-dodecyl-cyclohexyl urea (DCU), is preferred as an inhibitor, while N-cyclohexyl-N′-dodecylurea (CDU) is particularly preferred. Some compounds, such as dicyclohexylcarbodiimide (a lipophilic diinide), can decompose to an active urea inhibitor such as DCU. Any particular urea derivative or other compound can be easily tested for its ability to inhibit sEH by standard assays, such as those discussed herein. The production and testing of urea and carbamate derivatives as sEH inhibitors is set forth in detail in, for example, Morisseau et al., Proc Natl Acad Sci (USA) 96:8849-8854 (1999).

N-Adamantyl-N′-dodecyl urea (“ADU”) is both metabolically stable and has particularly high activity on sEH. (Both the 1- and the 2-admamantyl ureas have been tested and have about the same high activity as an inhibitor of sEH.) Thus, isomers of adamantyl dodecyl urea are particularly preferred inhibitors. It is further expected that other dodecanoic acid ester derivatives of urea are suitable for use in the methods of the invention.

U.S. Pat. No. 5,955,496 (the '496 patent) sets forth a number of suitable epoxide hydrolase inhibitors for use in the methods of the invention. One category of inhibitors comprises inhibitors that mimic the substrate for the enzyme. The lipid alkoxides (e.g., the 9-methoxide of stearic acid) are an exemplar of this group of inhibitors. In addition to the inhibitors discussed in the '496 patent, a dozen or more lipid alkoxides have been tested as sEH inhibitors, including the methyl, ethyl, and propyl alkoxides of oleic acid (also known as stearic acid alkoxides), linoleic acid, and arachidonic acid, and all have been found to act as inhibitors of sEH.

In another group of embodiments, the '496 patent sets forth sEH inhibitors that provide alternate substrates for the enzyme that are turned over slowly. Exemplars of this category of inhibitors are phenyl glycidols (e.g., S, S-4-nitrophenylglycidol), and chalcone oxides. The '496 patent notes that suitable chalcone oxides include 4-phenylchalcone oxide and 4-fluourochalcone oxide. The phenyl glycidols and chalcone oxides are believed to form stable acyl enzymes.

Additional inhibitors of sEH suitable for use in the methods of the invention are set forth in U.S. Pat. No. 6,150,415 (the '415 patent) and U.S. Pat. No. 6,531,506 (the '506 patent). Two preferred classes of inhibitors of the invention are compounds of Formulas 1 and 2, as described in the '415 and '506 patents. Means for preparing such compounds and assaying desired compounds for the ability to inhibit epoxide hydrolases are also described. The '506 patent, in particular, teaches scores of inhibitors of Formula 1 and some twenty inhibitors of Formula 2, which were shown to inhibit human sEH at concentrations as low as 0.1 μM. Any particular inhibitor can readily be tested to determine whether it will work in the methods of the invention by standard assays, such as that set forth in the Examples, below.

As noted above, chalcone oxides can serve as an alternate substrate for the enzyme. While chalcone oxides have half lives which depend in part on the particular structure, as a group the chalcone oxides tend to have relatively short half lives (a drug's half life is usually defined as the time for the concentration of the drug to drop to half its original value. See, e.g., Thomas, G., Medicinal Chemistry: an introduction, John Wiley & Sons Ltd. (West Sussex, England, 2000)). Since the uses of the invention contemplate inhibition of sEH over periods of time which can be measured in days, weeks, or months, chalcone oxides, and other inhibitors which have a half life whose duration is shorter than the practitioner deems desirable, are preferably administered in a manner which provides the agent over a period of time. For example, the inhibitor can be provided in materials that release the inhibitor slowly, including materials that release the inhibitor in or near the kidney, to provide a high local concentration. Methods of administration that permit high local concentrations of an inhibitor over a period of time are known, and are not limited to use with inhibitors which have short half lives although, for inhibitors with a relatively short half life, they are a preferred method of administration.

In addition to the compounds in Formula 1 of the '506 patent, which interact with the enzyme in a reversible fashion based on the inhibitor mimicking an enzyme-substrate transition state or reaction intermediate, one can have compounds that are irreversible inhibitors of the enzyme. The active structures such as those in the Tables or Formula 1 of the '506 patent can direct the inhibitor to the enzyme where a reactive functionality in the enzyme catalytic site can form a covalent bond with the inhibitor. One group of molecules which could interact like this would have a leaving group such as a halogen or tosylate which could be attacked in an SN2 manner with a lysine or histidine. Alternatively, the reactive functionality could be an epoxide or Michael acceptor such as an α/β-unsaturated ester, aldehyde, ketone, ester, or nitrile.

Further, in addition to the Formula 1 compounds, active derivatives can be designed for practicing the invention. For example, dicyclohexyl thio urea can be oxidized to dicyclohexylcarbodiimide which, with enzyme or aqueous acid (physiological saline), will form an active dicyclohexylurea. Alternatively, the acidic protons on carbamates or ureas can be replaced with a variety of substituents which, upon oxidation, hydrolysis or attack by a nucleophile such as glutathione, will yield the corresponding parent structure. These materials are known as prodrugs or protoxins (Gilman et al., The Pharmacological Basis of Therapeutics, 7th Edition, MacMillan Publishing Company, New York, p. 16 (1985)) Esters, for example, are common prodrugs which are released to give the corresponding alcohols and acids enzymatically (Yoshigae et al., Chirality, 9:661-666 (1997)). The drugs and prodrugs can be chiral for greater specificity. These derivatives have been extensively used in medicinal and agricultural chemistry to alter the pharmacological properties of the compounds such as enhancing water solubility, improving formulation chemistry, altering tissue targeting, altering volume of distribution, and altering penetration. They also have been used to alter toxicology profiles.

There are many prodrugs possible, but replacement of one or both of the two active hydrogens in the ureas described here or the single active hydrogen present in carbamates is particularly attractive. Such derivatives have been extensively described by Fukuto and associates. These derivatives have been extensively described and are commonly used in agricultural and medicinal chemistry to alter the pharmacological properties of the compounds. (Black et al., Journal of Agricultural and Food Chemistry, 21(5):747-751 (1973); Fahmy et al, Journal of Agricultural and Food Chemistry, 26(3):550-556 (1978); Jojima et al., Journal of Agricultural and Food Chemistry, 31(3):613-620 (1983); and Fahmy et al., Journal of Agricultural and Food Chemistry, 29(3):567-572 (1981).)

Such active proinhibitor derivatives are within the scope of the present invention, and the just-cited references are incorporated herein by reference. Without being bound by theory, it is believed that suitable inhibitors of the invention mimic the enzyme transition state so that there is a stable interaction with the enzyme catalytic site. The inhibitors appear to form hydrogen bonds with the nucleophilic carboxylic acid and a polarizing tyrosine of the catalytic site.

In some embodiments, sEH inhibition can include the reduction of the amount of sEH. As used herein, therefore, sEH inhibitors can therefore encompass nucleic acids that inhibit expression of a gene encoding sEH. Many methods of reducing the expression of genes, such as reduction of transcription and siRNA, are known, and are discussed in more detail below.

Preferably, the inhibitor inhibits sEH without also significantly inhibiting microsomal epoxide hydrolase (“mEH”). Preferably, at concentrations of 500 μM, the inhibitor inhibits sEH activity by at least 50% while not inhibiting mEH activity by more than 10%. Preferred compounds have an IC50 (inhibition potency or, by definition, the concentration of inhibitor which reduces enzyme activity by 50%) of less than about 500 μM. Inhibitors with IC50s of less than 500 μM are preferred, with IC50s of less than 100 μM being more preferred and IC50s of 50 μM, 40 μM, 30 μM, 25 μM, 20 μM, 15 μM, 10 μM, 5 μM, 3 μM, 2 μM, 1 μM or even less being the more preferred as the IC50 decreases. Assays for determining EH activity are known in the art and described elsewhere herein.

IV. EETs

EETs can be administered to inhibit damage from stroke. In preferred embodiments, one or more EETs are administered concurrently or after administration of an sEH inhibitor so that the EET or EETs are not hydrolyzed quickly.

Optionally, the EET or EETs are embedded or otherwise placed in a material that releases the EET over time. Materials suitable for promoting the slow release of compositions such as EETs are known in the art.

Conveniently, the EET or EETs can be administered orally. Since EETs are subject to degradation under acidic conditions, EETs intended for oral administration can be coated with a coating resistant to dissolving under acidic conditions, but which dissolve under the mildly basic conditions present in the intestines. Suitable coatings, commonly known as “enteric coatings” are widely used for products, such as aspirin, which cause gastric distress or which would undergo degradation upon exposure to gastric acid. By using coatings with an appropriate dissolution profile, the coated substance can be released in a chosen section of the intestinal tract. For example, a substance to be released in the colon is coated with a substance that dissolves at pH 6.5-7, while substances to be released in the duodenum can be coated with a coating that dissolves at pH values over 5.5. Such coatings are commercially available from, for example, Rohm Specialty Acrylics (Rohm America LLC, Piscataway, N.J.) under the trade name “Eudragit®”. The choice of the particular enteric coating is not critical to the practice of the invention.

Preferred EETs include 14,15-EET, 8,9-EET and 11,12-EET in that order of preference. Purified sEH selected 8S,9R- and 14R,15S-EET; accordingly these EETs are particularly preferred. 8,9-EET, 11,12-EET, and 14R,15S-EET are commercially available from, for example, Sigma-Aldrich (catalog nos. E5516, E5641, and E5766, respectively, Sigma-Aldrich Corp., St. Louis, Mo.).

V. Assays for Epoxide Hydrolase Activity

Any of a number of standard assays for determining epoxide hydrolase activity can be used to determine inhibition of sEH. For example, suitable assays are described in Gill, et al., Anal Biochem 131, 273-282 (1983); and Borhan, et al., Analytical Biochemistry 231, 188-200 (1995)). Suitable in vitro assays are described in Zeldin et al., J Biol. Chem. 268:6402-6407 (1993). Suitable in vivo assays are described in Zeldin et al., Arch Biochem Biophys 330:87-96 (1996). Assays for epoxide hydrolase using both putative natural substrates and surrogate substrates have been reviewed (see, Hammock, et al. In: Methods in Enzymology, Volume III, Steroids and Isoprenoids, Part B, (Law, J. H. and H. C. Rilling, eds. 1985), Academic Press, Orlando, Fla., pp. 303-311 and Wixtrom et al., In: Biochemical Pharmacology and Toxicology, Vol. 1: Methodological Aspects of Drug Metabolizing Enzymes, (Zakim, D. and D. A. Vessey, eds. 1985), John Wiley & Sons, Inc., New York, pp. 1-93. Several spectral based assays exist based on the reactivity or tendency of the resulting diol product to hydrogen bond (see, e.g., Wixtrom, supra, and Hammock. Anal. Biochem. 174:291-299 (1985) and Dietze, et al. Anal. Biochem. 216:176-187 (1994)).

The enzyme also can be detected based on the binding of specific ligands to the catalytic site which either immobilize the enzyme or label it with a probe such as dansyl, fluoracein, luciferase, green fluorescent protein or other reagent. The enzyme can be assayed by its hydration of EETs, its hydrolysis of an epoxide to give a colored product as described by Dietze et al., 1994, supra, or its hydrolysis of a radioactive surrogate substrate (Borhan et al., 1995, supra). The enzyme also can be detected based on the generation of fluorescent products following the hydrolysis of the epoxide. Numerous method of epoxide hydrolase detection have been described (see, e.g., Wixtrom, supra).

The assays are normally carried out with a recombinant enzyme following affinity purification. They can be carried out in crude tissue homogenates, cell culture or even in vivo, as known in the art and described in the references cited above.

VI. Other Means of Inhibiting sEH Activity

Other means of inhibiting sEH activity or gene expression can also be used in the methods of the invention. For example, a nucleic acid molecule complementary to at least a portion of the human sEH gene can be used to inhibit sEH gene expression. Means for inhibiting gene expression using, for example, short interfering RNA (siRNA), are known. “RNA interference”, a form of post-transcriptional gene silencing (“PTGS”), describes effects that result from the introduction of double-stranded RNA into cells (reviewed in Fire, A. Trends Genet 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown. While RNAi was shown initially to work well in lower eukaryotes, for mammalian cells, it was thought that RNAi might be suitable only for studies on the oocyte and the preimplantation embryo. In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of sEH, siRNAs to the gene encoding sEH can be specifically designed using computer programs. The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; nucleotides 42-1703 of SEQ ID NO:1 of that patent are the nucleic acid sequence encoding the amino acid sequence.

A program, siDESIGN from Dharmacon, Inc. (Lafayette, Colo.), permits predicting siRNAs for any nucleic acid sequence, and is available on the World Wide Web at dharmacon.com. Programs for designing siRNAs are also available from others, including Genscript (available on the Web at genscript.com/ssl-bin/app/rnai) and, to academic and non-profit researchers, from the Whitehead Institute for Biomedical Research on the internet by entering “http://” followed by “jura.wi.mit.edu/pubint/http://iona.wi.mit.edu/siRNAext/.”

For example, using the program available from the Whitehead Institute, the following sEH target sequences and siRNA sequences can be generated:

| | | | |
|---|---|---|---|
| 1) | Target: | CAGTGTTCATTGGCCATGACTGG | (SEQ ID NO:3) |
| | Sense-siRNA: | 5′ - GUGUUCAUUGGCCAUGACUTT- 3′ | (SEQ ID NO:4) |
| | Antisense-siRNA: | 5′ - AGUCAUGGCCAAUGAACACTT- 3′ | (SEQ ID NO:5) |
| 2) | Target: | GAAAGGCTATGGAGAGTCATCTG | (SEQ ID NO:6) |
| | Sense-siRNA: | 5′ - AAGGCUAUGGAGAGUCAUCTT - 3′ | (SEQ ID NO:7) |
| | Antisense-siRNA: | 5′ - GAUGACUCUCCAUAGCCUUTT - 3′ | (SEQ ID NO:8) |
| 3) | Target | AAAGGCTATGGAGAGTCATCTGC | (SEQ ID NO:9) |
| | Sense-siRNA: | 5′ - AGGCUAUGGAGAGUCAUCUTT- 3′ | (SEQ ID NO:10) |
| | Antisense-siRNA: | 5′ - AGAUGACUCUCCAUAGCCUTT- 3′ | (SEQ ID NO:11) |
| 4) | Target: | CAAGCAGTGTTCATTGGCCATGA | (SEQ ID NO:12) |
| | Sense-siRNA: | 5′ - AGCAGUGUUCAUUGGCCAUTT- 3′ | (SEQ ID NO:13) |
| | Antisense-siRNA: | 5′ - AUGGCCAAUGAACACUGCUTT- 3′ | (SEQ ID NO:14) |
| 5) | Target: | CAGCACATGGAGGACTGGATTCC | (SEQ ID NO:15) |
| | Sense-siRNA: | 5′ - GCACAUGGAGGACUGGAUUTT- 3′ | (SEQ ID NO:16) |
| | Antisense-siRNA: | 5′ - AAUCCAGUCCUCCAUGUGCTT- 3′ | (SEQ ID NO:17) |

Alternatively, siRNA can be generated using kits which generate siRNA from the gene. For example, the “Dicer siRNA Generation” kit (catalog number T510001, Gene Therapy Systems, Inc., San Diego, Calif.) uses the recombinant human enzyme “dicer” in vitro to cleave long double stranded RNA into 22 bp siRNAs. By having a mixture of siRNAs, the kit permits a high degree of success in generating siRNAs that will reduce expression of the target gene. Similarly, the Silencer™ siRNA Cocktail Kit (RNase III) (catalog no. 1625, Ambion, Inc., Austin, Tex.) generates a mixture of siRNAs from dsRNA using RNase III instead of dicer. Like dicer, RNase III cleaves dsRNA into 12-30 bp dsRNA fragments with 2 to 3 nucleotide 3′ overhangs, and 5′-phosphate and 3′-hydroxyl termini. According to the manufacturer, dsRNA is produced using T7 RNA polymerase, and reaction and purification components included in the kit. The dsRNA is then digested by RNase III to create a population of siRNAs. The kit includes reagents to synthesize long dsRNAs by in vitro transcription and to digest those dsRNAs into siRNA-like molecules using RNase III. The manufacturer indicates that the user need only supply a DNA template with opposing T7 phage polymerase promoters or two separate templates with promoters on opposite ends of the region to be transcribed.

The siRNAs can also be expressed from vectors. Typically, such vectors are administered in conjunction with a second vector encoding the corresponding complementary strand. Once expressed, the two strands anneal to each other and form the functional double stranded siRNA. One exemplar vector suitable for use in the invention is pSuper, available from OligoEngine, Inc. (Seattle, Wash.). In some embodiments, the vector contains two promoters, one positioned downstream of the first and in antiparallel orientation. The first promoter is transcribed in one direction, and the second in the direction antiparallel to the first, resulting in expression of the complementary strands. In yet another set of embodiments, the promoter is followed by a first segment encoding the first strand, and a second segment encoding the second strand. The second strand is complementary to the palindrome of the first strand. Between the first and the second strands is a section of RNA serving as a linker (sometimes called a “spacer”) to permit the second strand to bend around and anneal to the first strand, in a configuration known as a “hairpin.”

The formation of hairpin RNAs, including use of linker sections, is well known in the art. Typically, an siRNA expression cassette is employed, using a Polymerase III promoter such as human U6, mouse U6, or human H1. The coding sequence is typically a 19-nucleotide sense siRNA sequence linked to its reverse complementary antisense siRNA sequence by a short spacer. Nine-nucleotide spacers are typical, although other spacers can be designed. For example, the Ambion website indicates that its scientists have had success with the spacer TTCAAGAGA (SEQ ID NO:18). Further, 5-6 T's are often added to the 3′ end of the oligonucleotide to serve as a termination site for Polymerase III. See also, Yu et al., Mol Ther 7(2):228-36 (2003); Matsukura et al., Nucleic Acids Res 31(15):e77 (2003).

As an example, the siRNA targets identified above can be targeted by hairpin siRNA as follows. And if you would like to attack the same targets by short hairpin RNAs, produced by a vector (permanent RNAi effect) you would put sense and antisense strand in a row with a loop forming sequence in between and suitable sequences for an adequate expression vector to both ends of the sequence. The ends of course depend on the cutting sites of the vector. The following are non-limiting examples of hairpin sequences that can be cloned into the pSuper vector:

| | | | |
|---|---|---|---|
| 1) | Target: | CAGTGTTCATTGGCCATGACTGG | (SEQ ID NO:19) |
| | Sense strand: | 5′-GATCCCCGTGTTCATTGGCCATGACTTTCAAGAGAAGTCATGGCCAATGAACACTTTTT-3′ | (SEQ ID NO:20) |
| | Antisense strand: | 5′-AGCTAAAAAGTGTTCATTGGCCATGACTTCTCTTGAAAGTCATGGCCAATGAACACGGG -3′ | (SEQ ID NO:21) |
| 2) | Target: | GAAAGGCTATGGAGAGTCATCTG | (SEQ ID NO:22) |
| | Sense strand: | 5′-GATCCCCAAGGCTATGGAGAGTCATCTTCAAGAGAGATGACTCTCCATAGCCTTTTTTT -3′ | (SEQ ID NO:23) |
| | Antisense strand: | 5′-AGCTAAAAAAAGGCTATGGAGAGTCATCTCTCTTGAAGATGACTCTCCATAGCCTTGGG -3′ | (SEQ ID NO:24) |
| 3) | Target: | AAAGGCTATGGAGAGTCATCTGC | (SEQ ID NO:25) |
| | Sense strand: | 5′-GATCCCCAGGCTATGGAGAGTCATCTTTCAAGAGAAGATGACTCTCCATAGCCTTTTTT -3′ | (SEQ ID NO:26) |
| | Antisense strand: | 5′-AGCTAAAAAAGGCTATGGAGAGTCATCATCTCTTGAAAGATGACTCTCCATAGCCTGGG -3′ | (SEQ ID NO:27) |
| 4) | Target: | CAAGCAGTGTTCATTGGCCATGA | (SEQ ID NO:28) |
| | Sense strand: | 5′-GATCCCCAGCAGTGTTCATTGGCCATTTCAAGAGAATGGCCAATGAACACTGCTTTTTT -3′ | (SEQ ID NO:29) |
| | Antisense strand: | 5′-AGCTAAAAAAGCAGTGTTCATTGGCCATTCTCTTGAAATGGCCAATGAACACTGCTGGG -3′ | (SEQ ID NO:30) |
| 5) | Target: | CAGCACATGGAGGACTGGATTCC | (SEQ ID NO:31) |
| | Sense strand | 5′-GATCCCCGCACATGGAGGACTGGATTTTCAAGAGAAATCCAGTCCTCCATGTGCTTTTT -3′ | (SEQ ID NO:32) |
| | Antisense strand: | 5′-AGCTAAAAAGCACATGGAGGACTGGATTTCTCTTGAAAATCCAGTCCTCCATGTGCGGG -3′ | (SEQ ID NO:33) |

In addition to siRNAs, other means are known in the art for inhibiting the expression of antisense molecules, ribozymes, and the like are well known to those of skill in the art. The nucleic acid molecule can be a DNA probe, a riboprobe, a peptide nucleic acid probe, a phosphorothioate probe, or a 2′-O methyl probe.

Generally, to assure specific hybridization, the antisense sequence is substantially complementary to the target sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to the sEH gene is retained as a functional property of the polynucleotide. In one embodiment, the antisense molecules form a triple helix-containing, or “triplex” nucleic acid. Triple helix formation results in inhibition of gene expression by, for example, preventing transcription of the target gene (see, e.g., Cheng et al., 1988, J. Biol. Chem. 263:15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83:9591)

Antisense molecules can be designed by methods known in the art. For example, Integrated DNA Technologies (Coralville, Iowa) makes available a program on the internet which can be found by entering “http://”, followed by “biotools.idtdna.com/antisense/AntiSense.aspx”, which will provide appropriate antisense sequences for nucleic acid sequences up to 10,000 nucleotides in length. Using this program to analyze the sEH gene provides the following exemplar sequences:

| | |
|---|---|
| 1) UGUCCAGUGCCCACAGUCCU | (SEQ ID NO:34) |
| 2) UUCCCACCUGACACGACUCU | (SEQ ID NO:35) |
| 3) GUUCAGCCUCAGCCACUCCU | (SEQ ID NO:36) |
| 4) AGUCCUCCCGCUUCACAGA | (SEQ ID NO:37) |
| 5) GCCCACUUCCAGUUCCUUUCC | (SEQ ID NO:38) |

In another embodiment, ribozymes can be designed to cleave the mRNA at a desired position. (See, e.g., Cech, 1995, Biotechnology 13:323; and Edgington, 1992, Biotechnology 10:256 and Hu et al., PCT Publication WO 94/03596).

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules of the invention may be prepared by de novo chemical synthesis or by cloning. For example, an antisense RNA can be made by inserting (ligating) a sEH gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

It will be appreciated that the oligonucleotides can be made using nonstandard bases (e.g., other than adenine, cytidine, guanine, thymine, and uridine) or nonstandard backbone structures to provides desirable properties (e.g., increased nuclease-resistance, tighter-binding, stability or a desired Tm). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, phosphoramidates.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, 1996, Current Opinion in Neurobiology 6:629-634. Another subsequence, the h (hydrophobic) domain of signal peptides, was found to have similar cell membrane translocation characteristics (see, e.g., Lin et al., 1995, J. Biol. Chem. 270:14255-14258). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

VII. Therapeutic Administration

EETs and inhibitors of sEH can be prepared and administered in a wide variety of oral, parenteral and topical dosage forms. In preferred forms, compounds for use in the methods of the present invention can be administered by injection, that is, intravenously, intramuscularly, intracutaneously, subcutaneously, intraduodenally, or intraperitoneally. The sEH inhibitor or EETs, or both, can also be administered by inhalation, for example, intranasally. Additionally, the sEH inhibitors, or EETs, or both, can be administered transdermally. Accordingly, the methods of the invention permit administration of pharmaceutical compositions comprising a pharmaceutically acceptable carrier or excipient and either a selected inhibitor or a pharmaceutically acceptable salt of the inhibitor.

For preparing pharmaceutical compositions from sEH inhibitors, or EETs, or both, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution. Transdermal administration can be performed using suitable carriers. If desired, apparatuses designed to facilitate transdermal delivery can be employed. Suitable carriers and apparatuses are well known in the art, as exemplified by U.S. Pat. Nos. 6,635,274, 6,623,457, 6,562,004, and 6,274,166.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

A therapeutically effective amount of the sEH inhibitor, or EETs, or both, is employed in slowing or inhibiting damage from stroke. The dosage of the specific compound for treatment depends on many factors that are well known to those skilled in the art. They include for example, the route of administration and the potency of the particular compound. An exemplary dose is from about 0.001 μM/kg to about 100 mg/kg body weight of the mammal.

EETs are unstable, and can be converted to DHET in acidic conditions, such as those in the stomach. To avoid this, EETs can be administered intravenously or by injection. EETs intended for oral administration can be encapsulated in a coating that protects the EETs during passage through the stomach. For example, the EETs can be provided with a so-called “enteric” coating, such as those used for some brands of aspirin, or embedded in a formulation. Such enteric coatings and formulations are well known in the art. In some formulations, the EETs, or a combination of the EETs and an sEH inhibitor are embedded in a slow-release formulation to facilitate administration of the agents over time.

Without further elaboration, it is believed that one skilled in the art can, using the preceding description, practice the present invention to its fullest extent.

### EXAMPLES

The following examples are offered to illustrate, but not to limit the claimed invention.

### Example 1

The sEH inhibitor, adamantyl dodecanoic acid (AUDA) was administered orally to six-week-old male stroke prone spontaneously hypertensive rats (“SPSHR”). Plasma levels of AUDA at the end of the treatment period averaged 5.03±0.32 ng/ml and the urinary AUDA excretion rate was 111.6±24.4 ng/day.

After six weeks of treatment, cerebral ischemia was induced by middle cerebral artery occlusion using the intralumenal suture occlusion technique. Rats were anesthetized with sodium pentobarbital (50 mg/kg IP) and body temperature was maintained at 37° C. using a microprocessor-controlled heating pad and a rectal temperature probe. The skin over the skull was retracted and the bone was thinned using a low speed drill. A laser Doppler flow probe (Perimed) was glued to the skull 2 mm posterior and 6 mm lateral to the bregma to allow for confirmation of middle cerebral artery (“MCA”) occlusion. The MCA was then occluded using the thread occlusion model as described by Zea Longa et al. “Reversible middle cerebral artery occlusion without craniotomy in rats”, Stroke 20:84-91 (1989). The common carotid artery was exposed by a midline incision and the pterygopalatine artery was ligated and the branches of the external carotid artery were cauterized. A 3-0 monofilament thread with a rounded tip was introduced into the external carotid artery in a retrograde fashion. This was advanced cranially into the internal carotid artery to the origin of the MCA; at this point a dramatic reduction in the cerebral blood flow was observed using the laser Doppler. The monofilament thread was sutured in place and the rat allowed to recover. Six hours post occlusion the rats were anesthetized and decapitated and the brains removed and sectioned coronally at 2 mm intervals from the frontal pole. The brain slices were incubated for 20 minutes at 37° C. in a 2% solution of triphenyl tetrazolium hydrochloride (“TTC”), to identify the infarcted region, then fixed in paraformaldehyde (2%). Digital images of the brain slices were produced and the infarct volume was analyzed using a computerized image analysis system (NIH image, version 1.55). The percent of the hemisphere infarcted was measured using the Swanson equation to account for cerebral edema.

The SPSHR treated with AUDA had significantly smaller cerebral infarcts than the vehicle treated SPSHR (35.9±3.83 vs. 53.1±3.54% hemisphere infarcted, AUDA vs. vehicle p<0.01 n=6). This difference in infarct size occurred independently of changes in systolic blood pressure (193±2 vs. 197±6 mm Hg, AUDA vs. vehicle).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A use of an inhibitor of soluble epoxide hydrolase (“sEH”) for the manufacture of a medicament to reduce brain damage from stroke.

2. A use of claim 1, wherein said stroke is an ischemic stroke.

3. A use of claim 1, wherein said stroke is a hemorrhagic stroke.

4. A use of claim 1, wherein said inhibitor of sEH is selected from the group consisting of adamantyl dodecyl urea, adamantyl dodecanoic acid, N-cyclohexyl-N′-dodecyl urea (CDU) and N,N′-dicyclohexylurea (DCU).

5. A use of a cis-epoxyeicosatrienoic acid (“EET”) for the manufacture of a medicament to reduce brain damage from stroke.

6. A use of claim 5, wherein said EET is selected from the group consisting of 14,15-EET, 8,9-EET and 11,12-EET.

7. A use of a nucleic acid that inhibits expression of soluble epoxide hydrolase (“sEH”) for the manufacture of a medicament for reducing brain damage from stroke.

8. A use of claim 7, wherein the nucleic acid is a small interfering RNA.

9. A method of reducing brain damage from a stroke, comprising administering an inhibitor of soluble epoxide hydrolase (“sEH”) to a subject who has suffered a stroke.

10. A method of claim 9, wherein said sEH inhibitor is administered within 6 hours of said stroke.

11. A method of claim 9, wherein said sEH inhibitor is selected from the group consisting of adamantyl dodecyl urea, adamantyl dodecanoic acid, N-cyclohexyl-N′-dodecyl urea (CDU) and N,N′-dicyclohexylurea (DCU).

12. A method of claim 9, further comprising administering a cis-epoxyeicosatrienoic acid (“EET”) to said subject.

13. A method of claim 12, wherein said EET is selected from the group consisting of 14,15-EET, 8,9-EET and 11,12-BET.

14. A method of reducing brain damage from a stroke, said method comprising administering an inhibitor of soluble epoxide hydrolase (“sEH”) to a subject at risk of suffering a stroke.

15. A method of claim 14, wherein said subject is selected from the group consisting of: a person who has hypertension, a person who uses tobacco, a person who has carotid artery disease, a person who has peripheral artery disease, a person who has atrial fibrillation, a person who has had one or more transient ischemic attacks (TIAs), a person who has a high red blood cell count, a person who has sickle cell disease, a person who has high blood cholesterol, a person who is obese, a female who uses alcohol in excess of one drink a day, a male who uses alcohol in excess of two drinks a day, a person who uses cocaine, a person who has a family history of stroke, a person who has had a previous stroke or heart attack, a person who has diabetes, and a person who is 60 years or more of age.

16. A method of claim 14, wherein said sEH inhibitor is selected from the group consisting of adamantyl dodecyl urea, adamantyl dodecanoic acid, N-cyclohexyl-N′-dodecyl urea (CDU) and N,N′-dicyclohexylurea (DCU).

17. A method of claim 14, further comprising administering a cis-epoxyeicosatrienoic acid (“EET”) to said subject.

18. A method of claim 17, wherein said EET is selected from the group consisting of 14,15-EET, 8,9-EET and 11,12-EET.

19. A method of claim 14, wherein said sEH inhibitor is a nucleic acid which inhibits expression of a gene encoding sEH.

20. A method of claim 14, wherein said nucleic acid is a short interfering RNA (“siRNA”).


SEQUENCE LISTING (38 sequences)

SEQ ID NO: 1-2 - skipped (2 entries)

SEQ ID NO: 3 - DNA, 23 bp, Artificial Sequence
  Description of Artificial Sequence:soluble epoxide hydrolase (sEH) target sequence
cagtgttcattggccatgactgg

SEQ ID NO: 4 - DNA, 21 bp, Artificial Sequence
  Description of Combined DNA/RNA Molecule: soluble epoxide hydrolase (sEH) Sense-siRNA
  Description of Artificial Sequence:soluble epoxide hydrolase (sEH) Sense-siRNA
guguucauuggccaugacutt

SEQ ID NO: 5 - DNA, 21 bp, Artificial Sequence
  Description of Combined DNA/RNA Molecule: soluble epoxide hydrolase (sEH) Antisense-siRNA
  Description of Artificial Sequence:soluble epoxide hydrolase (sEH) Antisense-siRNA
agucauggccaaugaacactt

SEQ ID NO: 6 - DNA, 23 bp, Artificial Sequence
  Description of Artificial Sequence:soluble epoxide hydrolase (sEH) target sequence
gaaaggctatggagagtcatctg

SEQ ID NO: 7 - DNA, 21 bp, Artificial Sequence
  Description of Combined DNA/RNA Molecule: soluble epoxide hydrolase (sEH) Sense-siRNA
  Description of Artificial Sequence:soluble epoxide hydrolase (sEH) Sense-siRNA
aaggcuauggagagucauctt

SEQ ID NO: 8 - DNA, 21 bp, Artificial Sequence
  Description of Combined DNA/RNA Molecule: soluble epoxide hydrolase (sEH) Antisense-siRNA
  Description of Artificial Sequence:soluble epoxide hydrolase (sEH) Antisense-siRNA
gaugacucuccauagccuutt

SEQ ID NO: 9 - DNA, 23 bp, Artificial Sequence
  Description of Artificial Sequence:soluble epoxide hydrolase (sEH) target sequence
aaaggctatggagagtcatctgc

SEQ ID NO: 10 - DNA, 21 bp, Artificial Sequence
  Description of Combined DNA/RNA Molecule: soluble epoxide hydrolase (sEH) Sense-siRNA
  Description of Artificial Sequence:soluble epoxide hydrolase (sEH) Sense-siRNA
aggcuauggagagucaucutt

SEQ ID NO: 11 - DNA, 21 bp, Artificial Sequence
  Description of Combined DNA/RNA Molecule: soluble epoxide hydrolase (sEH) Antisense-siRNA
  Description of Artificial Sequence:soluble epoxide hydrolase (sEH) Antisense-siRNA
agaugacucuccauagccutt

SEQ ID NO: 12 - DNA, 23 bp, Artificial Sequence
  Description of Artificial Sequence:soluble epoxide hydrolase (sEH) target sequence
caagcagtgttcattggccatga

SEQ ID NO: 13 - DNA, 21 bp, Artificial Sequence
  Description of Combined DNA/RNA Molecule: soluble epoxide hydrolase (sEH) Sense-siRNA
  Description of Artificial Sequence:soluble epoxide hydrolase (sEH) Sense-siRNA
agcaguguucauuggccautt

SEQ ID NO: 14 - DNA, 21 bp, Artificial Sequence
  Description of Combined DNA/RNA Molecule: soluble epoxide hydrolase (sEH) Antisense-siRNA
  Description of Artificial Sequence:soluble epoxide hydrolase (sEH) Antisense-siRNA
auggccaaugaacacugcutt

SEQ ID NO: 15 - DNA, 23 bp, Artificial Sequence
  Description of Artificial Sequence:soluble epoxide hydrolase (sEH) target sequence
cagcacatggaggactggattcc

SEQ ID NO: 16 - DNA, 21 bp, Artificial Sequence
  Description of Combined DNA/RNA Molecule: soluble epoxide hydrolase (sEH) Sense-siRNA
  Description of Artificial Sequence:soluble epoxide hydrolase (sEH) Sense-siRNA
gcacauggaggacuggauutt

SEQ ID NO: 17 - DNA, 21 bp, Artificial Sequence
  Description of Combined DNA/RNA Molecule: soluble epoxide hydrolase (sEH) Antisense-siRNA
  Description of Artificial Sequence:soluble epoxide hydrolase (sEH) Antisense-siRNA
aauccaguccuccaugugctt

SEQ ID NO: 18 - DNA, 9 bp, Artificial Sequence
  Description of Artificial Sequence:short spacer linking sense siRNA and reverse complementary antisense siRNA
ttcaagaga

SEQ ID NO: 19 - DNA, 23 bp, Artificial Sequence
  Description of Artificial Sequence:hairpin siRNA target
cagtgttcattggccatgactgg

SEQ ID NO: 20 - DNA, 59 bp, Artificial Sequence
  Description of Artificial Sequence:hairpin siRNA Sense strand
gatccccgtgttcattggccatgactttcaagagaagtcatggccaatgaacacttttt

SEQ ID NO: 21 - DNA, 59 bp, Artificial Sequence
  Description of Artificial Sequence:hairpin siRNA Antisense strand
agctaaaaagtgttcattggccatgacttctcttgaaagtcatggccaatgaacacggg

SEQ ID NO: 22 - DNA, 23 bp, Artificial Sequence
  Description of Artificial Sequence:hairpin siRNA target
gaaaggctatggagagtcatctg

SEQ ID NO: 23 - DNA, 59 bp, Artificial Sequence
  Description of Artificial Sequence:hairpin siRNA Sense strand
gatccccaaggctatggagagtcatcttcaagagagatgactctccatagccttttttt

SEQ ID NO: 24 - DNA, 59 bp, Artificial Sequence
  Description of Artificial Sequence:hairpin siRNA Antisense strand
agctaaaaaaaggctatggagagtcatctctcttgaagatgactctccatagccttggg

SEQ ID NO: 25 - DNA, 23 bp, Artificial Sequence
  Description of Artificial Sequence:hairpin siRNA target
aaaggctatggagagtcatctgc

SEQ ID NO: 26 - DNA, 59 bp, Artificial Sequence
  Description of Artificial Sequence:hairpin siRNA Sense strand
gatccccaggctatggagagtcatctttcaagagaagatgactctccatagcctttttt

SEQ ID NO: 27 - DNA, 59 bp, Artificial Sequence
  Description of Artificial Sequence:hairpin siRNA Antisense strand
agctaaaaaaggctatggagagtcatcatctcttgaaagatgactctccatagcctggg

SEQ ID NO: 28 - DNA, 23 bp, Artificial Sequence
  Description of Artificial Sequence:hairpin siRNA target
caagcagtgttcattggccatga

SEQ ID NO: 29 - DNA, 59 bp, Artificial Sequence
  Description of Artificial Sequence:hairpin siRNA Sense strand
gatccccagcagtgttcattggccatttcaagagaatggccaatgaacactgctttttt

SEQ ID NO: 30 - DNA, 59 bp, Artificial Sequence
  Description of Artificial Sequence:hairpin siRNA Antisense strand
agctaaaaaagcagtgttcattggccattctcttgaaatggccaatgaacactgctggg

SEQ ID NO: 31 - DNA, 23 bp, Artificial Sequence
  Description of Artificial Sequence:hairpin siRNA target
cagcacatggaggactggattcc

SEQ ID NO: 32 - DNA, 59 bp, Artificial Sequence
  Description of Artificial Sequence:hairpin siRNA Sense strand
gatccccgcacatggaggactggattttcaagagaaatccagtcctccatgtgcttttt

SEQ ID NO: 33 - DNA, 59 bp, Artificial Sequence
  Description of Artificial Sequence:hairpin siRNA Antisense strand
agctaaaaagcacatggaggactggatttctcttgaaaatccagtcctccatgtgcggg

SEQ ID NO: 34 - RNA, 20 nt, Artificial Sequence
  Description of Artificial Sequence:soluble epoxide hydrolase (sEH) exemplar antisense sequence
uguccagugcccacaguccu

SEQ ID NO: 35 - RNA, 20 nt, Artificial Sequence
  Description of Artificial Sequence:soluble epoxide hydrolase (sEH) exemplar antisense sequence
uucccaccugacacgacucu

SEQ ID NO: 36 - RNA, 20 nt, Artificial Sequence
  Description of Artificial Sequence:soluble epoxide hydrolase (sEH) exemplar antisense sequence
guucagccucagccacuccu

SEQ ID NO: 37 - RNA, 19 nt, Artificial Sequence
  Description of Artificial Sequence:soluble epoxide hydrolase (sEH) exemplar antisense sequence
aguccucccgcuucacaga

SEQ ID NO: 38 - RNA, 21 nt, Artificial Sequence
  Description of Artificial Sequence:soluble epoxide hydrolase (sEH) exemplar antisense sequence
gcccacuuccaguuccuuucc